(19) United States
(12) Patent Application Publication
(10) Pub. No.: US 2019/0374556 A1
(43) Pub. Date: Dec. 12, 2019

(54) COMPOSITIONS AND METHODS FOR KELOIDLESS HEALING

(71) Applicants: Masayo AOKI, Tokyo (JP); Health Research, Inc., Buffalo, NY (US)

(72) Inventors: Kazuaki TAKABE, Clarence, NY (US); Masayo Aoki, Tokyo (JP)

(21) Appl. No.: 16/483,360

(22) PCT Filed: Feb. 2, 2018
(86) PCT No.: PCT/US2018/016564

§ 371 Date: Aug. 2, 2019

Related U.S. Application Data
(60) Provisional application No. 62/453,845, filed on Feb. 2, 2017.

(51) Int. Cl. A61K 31/661; A61K 38/46; A61K 48/00; A61K 9/06; A61K 9/00; A61K 9/70; A61P 17/02; A61K 47/69

(52) U.S. Cl. CPC A61K 31/661; A61K 38/465; A61K 48/0033; A61K 47/6923; A61K 9/0014; A61K 9/7007; A61P 17/02; A61K 9/06

(57) ABSTRACT

Provided are compositions, methods and devices for reducing scarring during healing of a tissue wound. The compositions and methods involve use of sphingosine-1-phosphate (S1P), and/or an expression vector that encodes sphingosine kinasel (SphK1). The compositions can be combined with other agents and implements, such as biocompatible nanoparticles, and medical devices involved with promoting wound healing. The approaches can reduce formation or prevent the occurrence of keloids.

### CROSS REFERENCE TO RELATED APPLICATIONS

This application claims priority to U.S. provisional patent application No. 62/453,845, filed Feb. 2, 2017, the disclosure of which is incorporated herein by reference.

### FIELD

The present disclosure relates generally to wound healing, and more specifically to the use of sphingosine-1-phosphate and/or expression vectors that encode sphingosine kinasel to inhibit scar formation.

### BACKGROUND

The process of wound healing includes three phases; inflammatory, proliferative, and remodeling phases¹⁸. In inflammatory phase, inflammatory cells are recruited into the wound and purification occurs³⁹. Further, inflammatory cells also play important roles with secretion of various kinds of wound-related factor in proliferative phase¹⁰. Current topical wound treatments including prostaglandin E1 or basic fibroblast growth factor fail to supply the full spectrum of wound-related factors, which is required to accelerate wound closure. However, there is an ongoing and unmet need for improved compositions and methods to promote wound healing, and particularly to inhibit the formation of scar tissue and/or keloids. The present disclosure is pertinent to these needs.

### SUMMARY OF THE DISCLOSURE

Embodiments of this disclosure comprises applying an effective amount of a composition comprising S1P or an expression vector that expresses SphK1 to a wound such that scar formation is inhibited, and/or keloid formation is inhibited, and/or keloidless healing of a wound occurs. In one aspect the disclosure comprises a method for reducing scarring during healing of a tissue wound comprising topically applying to the wound a composition comprising sphingosine-1-phosphate (S1P), and/or an expression vector that encodes sphingosine kinasel (SphK1). In embodiments, the composition comprises the expression vector, further comprises biocompatible nanoparticles, including but not limited to nanoparticles formed with super carbonate apatite (sCA).

In embodiments, scarring in the wound is reduced relative to a control, wherein the control comprises a value from wound healing in the absence of exogenously applied S1P and/or an absence of the expression vector. In certain implementations, methods of this disclosure result in inhibition or prevention of keloid formation. The compositions can be provided in any suitable formulation, one non-limiting example of which comprises an ointment. The compositions can be administered using any suitable route, one non-limiting example of which comprises topical administration.

In another aspect the disclosure provides an article of manufacture comprising a composition as described herein, the article comprising packaging, the packaging comprising printed material providing instructions for using the composition and an indication that the composition is for use in healing of wounds.

In another aspect the compositions are coated onto and/or integrated into a device, not limiting examples of which include a wound dressing, a suture, and a staple.

### BRIEF DESCRIPTION OF THE FIGURES

FIG. 1. Sphingolipid metabolism in mouse wound closure process. (a) SphK1, (b) SphK2, and (c) S1PR1/2 mRNA expression in mouse wound closure process (n=4-6). (d) Wound area analysis in SphK1 WT vs. KO mice (n=6-10). (e) Flow cytometry analysis for T cell population in SphK1 WT vs. KO mice at day 5 after punch (n=5). (f) Representative immunohistochemistry for F4/80 and (g) percentage of F4/80 positive area at day 5 after punch. Arrowheads indicate macrophages (scale bars: 50 μm, n=4). (h) Representative immunohistochemistry for Ki67 and (i) percentage of Ki67 positive cells area at day 5 after punch. Arrowheads indicate Ki67 positive cells (scale bars: 50 μm, n=4). (j) Representative immunohistochemistry for CD34 and (k) numbers of microvessels per 200-fold magnified field at day 5 after punch. Arrowheads indicate microvessels (scale bars: 100 μm, n=4). (1) Wound area analysis in S1PR2 WT vs. KO mice (n=6). Values are means±s.e.m. *p<0.05, **p<0.01.

FIG. 2. S1P treatment promotes wound closure with increased macrophage recruitment and angiogenesis. (a) Representative photo images in mouse wound closure in vehicle vs. 1 μM S1P topical treatment. (b) Wound area analysis in vehicle vs. 1 μM S1P topical treatment (n=12). (c) Flow cytometry analysis for T cell population in vehicle vs. 1 μM S1P topical treatment at day 5 after punch (n=5). (d) Representative immunohistochemistry for F4/80 and (e) Percentage of F4/80 positive area at day 5 after punch. Arrowheads indicate macrophages (scale bars: 50μm, n=4). (f) Representative immunohistochemistry for Ki67 and (g) percentage of Ki67 positive cells area at day 5 after punch. Arrowheads indicate Ki67 positive cells (scale bars: 50 μm, n=4). (h) Representative immunohistochemistry for CD34 and (i) numbers of microvessels per 200-fold magnified field at day 5 after punch. Arrowheads indicate microvessels (scale bars: 100 μm, n=4). (j) Representative photo-acoustic images in wounds of Balb/c mice at day 6 after punch. (k) Quantitated microvascular integrated density of photo-acoustic images (n=11). (l) Flow cytometry analysis for CD31 positive CD45 negative cells at day 7 (n=12). Values are means s.e.m. *p<0.05, **p<0.01.

FIG. 3. Nanoparticle-mediated topical SphK1 gene delivery promotes wound closure with increased inflammatory cell recruitment and production of various wound-related factors. (a) Preparation of SphK1 expressing plasmid-capsuled sCA ointment. (b) Immunoblots for V5-SphK1 in wound surface tissues at 2 day after application. (c) Representative photo images in mouse wound closure in vector vs. SphK1-sCA topical treatment. (d) Wound area analysis in vector vs. SphK1-sCA topical treatment (n=12). (e) Flow cytometry analysis for T cell population in vector vs. SphK1-sCA topical treatment at day 5 after punch (n=6-8). (f) Representative immunohistochemistry for F4/80 and (g) percentage of F4/80 positive area at day 5 after punch. Arrowheads indicate macrophages (scale bars: 50 μm, n=4). (h) Representative immunohistochemistry for Ki67 and (i) percentage of Ki67 positive cells area at day 5 after punch. Arrowheads indicate Ki67 positive cells (scale bars: 50 μm, n=4). (j) Representative immunohistochemistry for CD34 and (k) numbers of microvessels per 200-fold magnified field at day 5 after punch.

Arrowheads indicate microvessels (scale bars: 100 μm, n=4). (1) Immunoblots for various wound-related factors in wounds at day 5 after punch. Values are means±s.e.m. *p<0.05, **p<0.0

FIG. 4. Topical SphK1 gene delivery induces scarless wound healing. (a) Representative Masson's trichrome images in scar at the point of epithelization in vehicle vs. 1 μM S1P topical treatment (scale bars: 400 μm). (b) Scar thickness in vehicle vs. S1P treatment (n=4-6). (c) Col1a1/Col3a1 mRNA expressions in NIH 3T3 cells stimulated with indicated concentration of S1P for 24 hours (n=4). (d) Col1a1/Col3a1 mRNA expressions in NIH 3T3 cells transfected with vector vs. SphK1 (n=4). (e) Col1a1/Col3a1 mRNA expressions in NIH 3T3 cells stimulated with 1 μM of S1P for 24 hours with or without 10 μM of VPC23019 or 10 μM of JTE013 (n=3). (f) S1PRs mRNA expressions in NIH 3T3 cells stimulated with indicated concentration of TGFβ-1 for 18 hours (n=3). (g) Schematic of TGFβ-1 and S1PR signaling in collagen transcription in dermal fibroblast. (h) Relative mRNA normalized by GAPDH. Values are means±s.e.m. *p<0.05, **p<0.01.

FIG. 5. 100 μM S1P topical treatment induces delayed wound closure. Wound area analysis treated with vehicle (BSA), 1 μM SIP, or 100 μM S1P ointments (a) in C57BL6/J (n=6), or (b) in Balb/c mice. (n=4-6). Values are means±s.e.m. *p<0.05, **p<0.01.

FIG. 6. Representative Neovasculature images in scar of Balb/c mice at the point of epithelization with vehicle vs. S1P treatment.

FIG. 7. In vitro transfection efficiency with SphK1 expressing plasmid using super carbonate apatite in various cells. (a) NIH 3T3 cells. (b) Hela cells. (c) HEK 293 cells (n=3). Values are means±s.e.m. *p<0.05, **p<0.01.

FIG. 8. S1P does not influence in recruitment of granulocytes in inflammatory phase of wound healing. Flow cytometry analysis for Gr-1 positive cells at day 2 after punch, (a) in SphK1 WT vs. KO mice (n=5), (b) in vehicle vs. 1 μM S1P topical treatment (n=5), (c) in vector vs. SphK1-sCA topical treatment (n=6-8). Values are means±s.e.m.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The present disclosure includes all DNA sequences, sequences complementary thereto, and all mRNA sequences encoded by the DNA sequences.

The present disclosure is related generally to the discovery that sphingosine-1-phosphate (S1P), and/or expression vectors that encode sphingosine kinasel (SphK1) which synthesizes S1P, inhibits scar formation during wound healing. Thus, the disclosure comprises administering a composition comprising S1P, and/or and an expression vector encoding SphK1, to a wound such that scar formation during healing of the wound is inhibited. In embodiments, inhibition of scar formation comprises reducing collagen production, thereby inhibiting excessive scaring known in the art as keloid formation. The disclosure in certain aspect is therefore directed to reducing keloid formation, and in certain implementations the disclosure results in keloidless healing of a wound. In embodiments performance of methods of this disclosure increase angiogenesis and/or proximal to a wound site.

In connection with keloids, it is known in the art that keloid scars are proliferative dermal growths that develop after skin injury. Without intending to be constrained by any particular theory, these benign dermal fibroproliferative tumors are made of type I and type Ill collagen, and occur in 5-15% of wounds, with an average age of onset between 10 to 30 years. Furthermore, they occur 15 times more frequently in persons with highly pigmented skin, than in persons of less pigmentation. Keloid scars can range from mildly cosmetically disfiguring to severely debilitating. Unlike hypertrophic scars, the scar tissue extends beyond the borders of the original wound. These unsightly, lumpy scars can form on any part of the body, and can grow quite large. Additionally, keloid scars can become inflamed and very painful. In these cases, inflammation develops and the pain is typically not alleviated until the inflammation subsides. A keloid scar in an area that is continually irritated, for example near the waistline, can cause persistent pain, with the keloid scar enlarging and hardening over time. In those affected by keloid scar formation, should a surgical procedure become necessary, for example removal of a skin cancer, the excision itself serves as the injury that stimulates keloid scar formation.

Certain non-limiting illustrations of the invention are shown using S1P as a composition of matter that is applied to a wound. Other equally non-limiting illustrations demonstrate applying plasmids encoding SphK1. Thus, the disclosure pertains to contacting a wound either directly with S1P, or by introducing an expression vector encoding SphK1 into cells proximal or within wounded tissue. In embodiments it is preferable to use an expression vector that expresses SphK1.

S1P is known in the art and it can be obtained commercially. The DNA sequence encoding murine and human forms of SphK1 are known in the art. The sequence encoding the human SphK1 gene can be accessed via Gene Card ID 8877. Any isoform of the SphK1 gene can be used. In this regard, there are three isoforms of human SphK1 protein produced by four splice variants, the amino acid sequences for which are available under accession numbers NP_068807.2, NP_892010.2 and NP_001136073. There are three isoforms of murine SphK1 from 5 splice variants. The present disclosure uses for non-limiting demonstrations the SphK1 variant 5, the GenBank accession number for which is NM_001172475.1 The GenBank accession number for murine isoform is NP_001165946.1 and has 83% homology with human SphK1 isoform 1-3, equally. Each of the polynucleotide sequences and amino acid sequences for each of these GenBank entries are incorporated herein as they exist on the effective filing date of this application or patent. The disclosure further comprises every polynucleotide sequence encoding these amino acid sequences, including polynucleotide sequences that are optimized for expression in any cell type, including but not limited to human cells. The disclosure includes all amino acid sequences that are between 80-99.9% similar to those in the stated database entries.

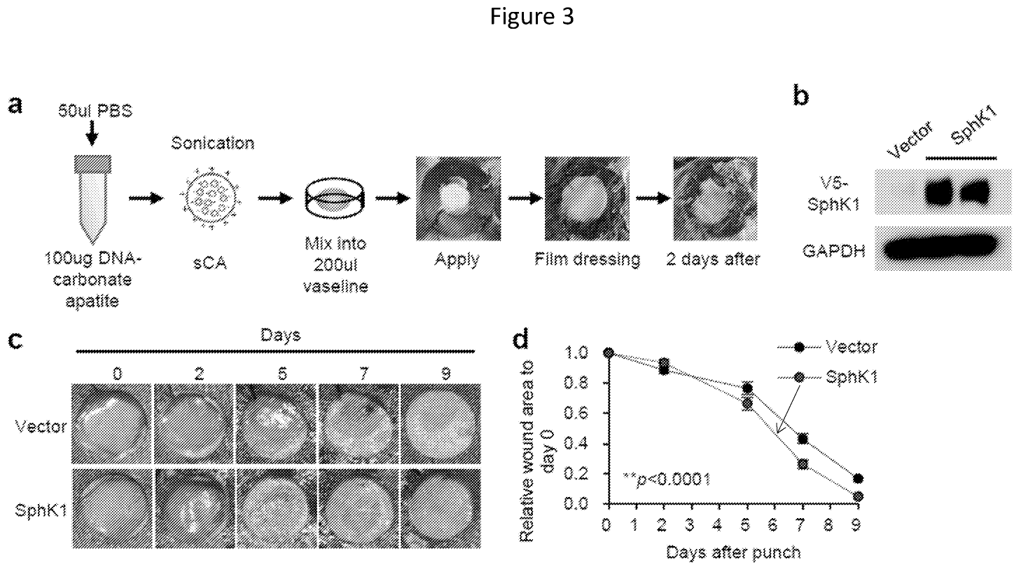

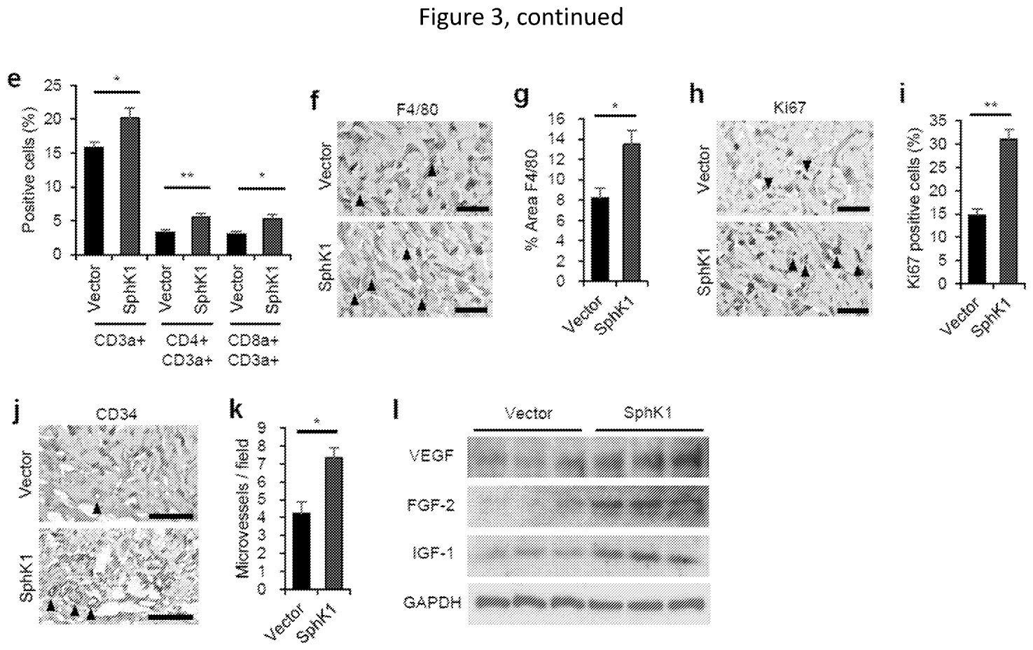

Any suitable expression vector can be adapted for SphK1 expression by inserting an SphK1-coding region into the plasmid such that S1P is produced by cells into which the expression vector is introduced. Thus, applying an expression vector to a wound is a manner of contacting a wound site with S1P produced by cells that express the SphK1. In general the expression vector, such as a DNA plasmid, is configured such that it cannot integrate into the host genome, but the plasmid expresses SphK1 for an adequate duration such that sufficient S1P is produced to reduce scarring and/or and promote keloidless healing. In certain approaches the SphkKi expression is expressed constitutively from, for example, a strong promoter. In a non-limiting example, the data presented in FIG. 3b were obtained after 2 days from the initial application of the expression plasmid to wounds.

In certain approaches the disclosure comprises applying an effective amount of a composition comprising S1P or an expression vector that expresses SphK1 to a wound such that scar formation is inhibited, and/or keloid formation is inhibited, and/or keloidless healing of a wound occurs. The wound can be to any part of an individual. In embodiments, the wound is in a soft tissue, such as skin, or is in an organ, for example, kidney or heart (myocardium infarction), or a muscle. In embodiments, the wound comprises an incision or other separation of tissue, or comprises a burn, or comprises a laceration, or an ulceration, such as a diabetic ulceration. In embodiments, the wound is caused by medical techniques such as surgical interventions wherein the skin, other tissue or an organ is cut or pierced or avulsed, or other non-medical wounds which cause trauma by any means, including but not necessarily to the accidental or intentional wounding of an individual, such as in a military conflict or other act of violence, an industrial accident, a vehicular accident, or an injury sustained during a sporting event. In certain embodiments the disclosure encompasses healing of wounds that are incidental to or a component of organ and/or tissue transplantation. In addition to wounds, the disclosure includes reducing scarring and/or keloid formation in any of numerous dermatologic diseases and conditions that are associated with keloid formation, among which are dissecting cellulitis of the scalp, acne vulgaris, acne conglobata, hidradenitis suppurativa, pilonidal cysts, foreign body reaction, and local infections with herpes, smallpox, or vaccinia. Keloids have also been observed in individual cases of patients with Ehlers-Danlos syndrome, Rubinstein-Taybi syndrome, pachydermoperiostosis, and epidermolysis bullosa.

Various methods known to those skilled in the art may be used to administer compositions of this disclosure. These methods include but are not necessarily limited to intradermal, transdermal, and subcutaneous routes. In certain aspects the disclosure includes providing the compositions in the form of creams, aqueous solutions, suspensions or dispersions, oils, balms, foams, lotions, gels, cream gels, hydrogels, liniments, serums, films, ointments, sprays or aerosols, other forms of coating, or any multiple emulsions, slurries or tinctures. In embodiments, a suitable ointment is prepared using any of a variety of well-known techniques and agents. In a non-limiting approach, a suitable ointment is prepared by using fat, fatty oil, lanolin, wax, resin, plastic, glycol, a high molecular alcohol, glycerin, water, an emulsifying agent, a suspending agent or other suitable excipient as a starting material and mixing it with an active ingredient described herein, or by using these ingredients as base ingredients and homogenously mixing them with an active ingredient, such as an expression vector and/or SP1. The base ingredients can be melted under heating and stirred homogenously.

The formulations of various embodiments may include any number of additional components such as, for example, preservatives, emulsion stabilizers, solubilizing agents, pH adjusters, chelating agents, viscosity modifiers, anti-oxidants, surfactants, emollients, opacifying agents, skin conditioners, buffers, fragrances, and combinations thereof. In some embodiments, such additional components may provide a dual purpose. For example, certain surfactants may also act as emulsifiers, certain emollients may also act as viscosity modifiers, and certain buffering agents may also act as chelating agents. In embodiments the compositions are provided as an oil-in-water emulsion. Thus, compositions of this disclosure can comprise additional components, such as antibiotics and other agents used to promote and/or aid in wound healing, such as antiseptic agents, and/or topical anesthetic agents. The compositions can further include other ingredients, such as proteins, free amino acids, humectants, essential oils, colorants, hydroxyacids, plant extracts, sunscreens, hyaluronate, lipids, fatty acids, thickeners, panthenol, and the like. Compositions may be formulated in a conventional manner using one or more physiologically acceptable carriers, diluents, excipients, or auxiliaries, and one or more pharmaceutically acceptable vehicles into formulations that can be used pharmaceutically.

The compositions may be embedded in materials, such as a medical device or other implement used in treating or manipulating a body, organ, or tissue. The compositions can also include liposomes, microsomes, nanoparticles, and any other suitable vehicle for delivering the compositions. In certain embodiments compositions of the disclosure comprise one or more biodegradable polymers. In general such polymers will degrade and be absorbed/cleared by the body after they have fulfilled their desired functions. U.S. Food and Drug Administration (FDA) approved aliphatic polyesters, such as poly(lactic acid) (PLA), poly(glycolic acid) (PGA), and their copolymers (PLGA) can be used, for example. In one approach super carbonate apatite (sCA) is used in compositions comprising an expression vector encoding SphK1 or S1P. sCA is known in the art to be comprised of inorganic ions, generally CO₃²⁻, Ca²⁺, and PO₄³⁻. In certain approaches an sCA preparation can be treated to reduce its particle size, such as by for example, sonication. In embodiments sCA can be used in a nanoparticle size that ranges in average diameter to from about 5 to 30 nm.

The compositions of this disclosure can be incorporated into devices and other articles that come into contact with and/or are intended to be used in conjunction with wounds, including but not necessarily limited to wound dressings, bandages, etc., as well as medical devices that can create injuries to the dermis, and further can be included in or with wound closure implements, such as sutures, staples, and other wound closure articles that will be apparent to those skilled in the art.

Given the benefit of the present disclosure, those skilled in the art will be able to determine an effective amount of compositions of this disclosure. Such determinations will be based on factors that can include but are not limited to the size, age and type of individual to be treated, and the type, size, severity, length, depth, type of tissue and/or location of the wound. However, it is demonstrated herein that increasing the amount of S1P can reduce efficacy to the point where the S1P application is not better than a control. Thus, in embodiments less than 100 μM S1P is used. In embodiments, from 0.1 μM -50.0 μM is used. In embodiments, from 0.1 μM-10.0 μM is used. In one embodiment, from 0.1 μM-2.0 μM is used. In embodiments, from 0.1 μM-1.0 μM is used. In one approach about 1.0 μM is used.

With respect to expression vectors, in various non-limiting demonstrations of this disclosure, the average (±SD) copy number at 2 day after transfection in a 5 mm wound is 3.82 (±2.64)×10⁹/wound. Expression is driven by either CMV or SV40 promoters.

In embodiments, the compositions and methods described herein are suitable for use with any mammal in need thereof. The mammal can be a human or a non-human mammal. Thus, in addition to human medicaments and treatment modalities, the present disclosure also encompasses veterinary aspects for the treatment of, for example, companion animals, livestock, etc.

In one embodiment, the disclosure includes an article of manufacture. In certain aspects, the article of manufacture includes a closed or sealed container, and packaging, that contains the compositions described herein. The package can include one or more containers, such as closed or sealed vials, bottles, and any other suitable packaging for the sale, or distribution, or use of pharmaceutical or biologic agents, such as expression vectors encoding SphK1. In addition to the pharmaceutical compositions, the package and/or container may contain printed information. The printed information can be provided on a label, or on a paper insert, or printed on the packaging material or container itself. The printed information can include information that identifies the ingredients, what the contents are intended to treat, and instructions for preparing the composition for administration, and/or for administering the composition to a wound. In certain embodiments the printed information can indicate that the compositions or prescribed by a health care provider, or they are for over-the-counter products.

The following Examples illustrate various aspects of this disclosure but are not intended to be limiting.

S1P signaling and recruitment of inflammatory cells and angiogenesis

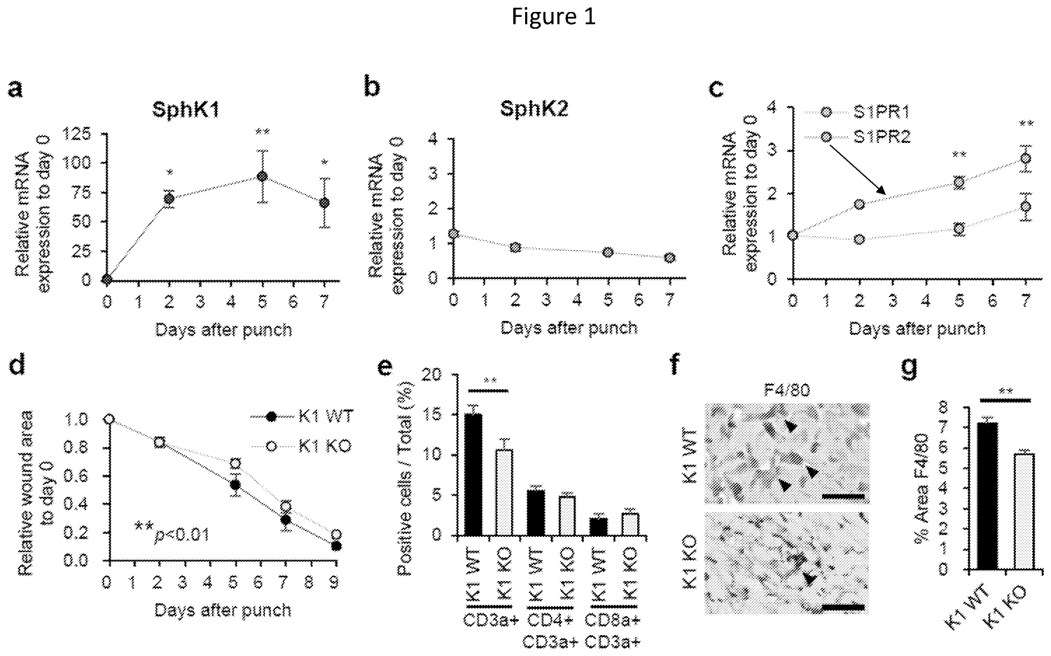

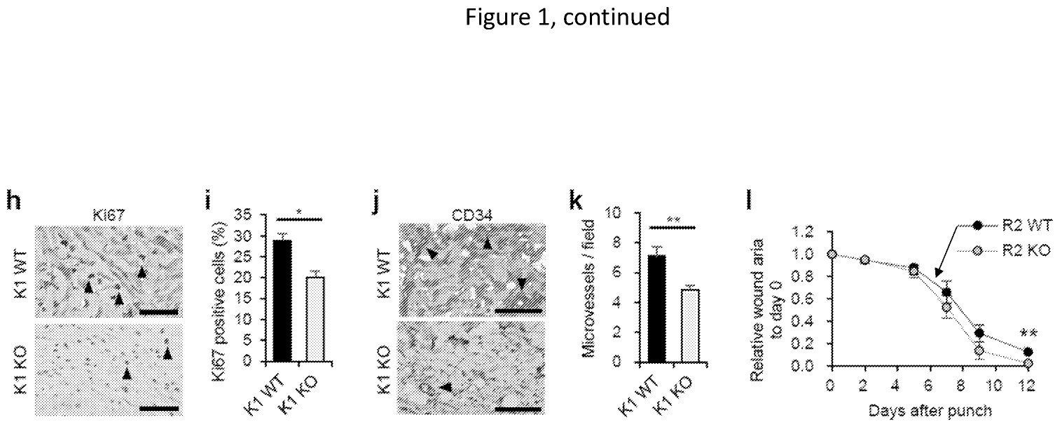

First, we investigated the role of S1P signaling during the mouse wound healing process. Expression of SphK1 in the wound demonstrated a significant increase from day 2 up to 88.6-fold increase at day 5 after injury (FIG. 1a), whereas there was no change in expression of SphK2 (FIG. 1b). Interestingly, expression of S1PR2 gradually increased during wound healing, where there was no change in expression of S1PR1 (FIG. 1c). These results show strong involvement of SphK1 in proliferative phase in particular, and indicate that it is not the activation of S1PR but the production of S1P by SphK1 that may be important for wound closure.

We next investigated the role of SphK1 during wound closure utilizing SphK1 knockout (KO) mice. Wound healing in SphK1 KO mice were significantly delayed compared with littermate wildtype (WT) (FIG. 1d). Flow cytometry analysis demonstrated that the percentage of CD3a⁺ cells in the wound was significantly lower in KO mice comparing with that in WT mice 5 days after injury (FIG. 1e). Despite the fact that blood S1P levels of SphK1 KO mice are about half of that of WT mice, lymphocyte trafficking has been report to remain intact because S1P concentration gradient between blood and second lymphoid organs is maintained²⁵. Our result demonstrated that lymphocyte recruitment into wound was clearly impaired in SphK1 KO mice. Furthermore, recruitment of macrophages (FIG. 1f,g), cell proliferation (FIG. 1h,i), and angiogenesis (FIG. 1j,k)are all suppressed in KO mice compared with WT mice. Recruited lymphocytes are the source of various kinds of wound-related factors, and play an important role in the process of proliferative phase.

Given the results that expression of S1PR2 in the wound increased during wound healing process, analyzed whether S1PR2 was involved with the wound closure process in S1PR2 KO mice. Although statistically significant differences were not detected with two factor repeated-measures ANOVA, the sizes of wounds at day 12 after injury were significantly smaller in S1PR2 KO mice in comparison with that in WT mice (FIG. 11). S1PR2 signaling results in negative effects for wound closure.

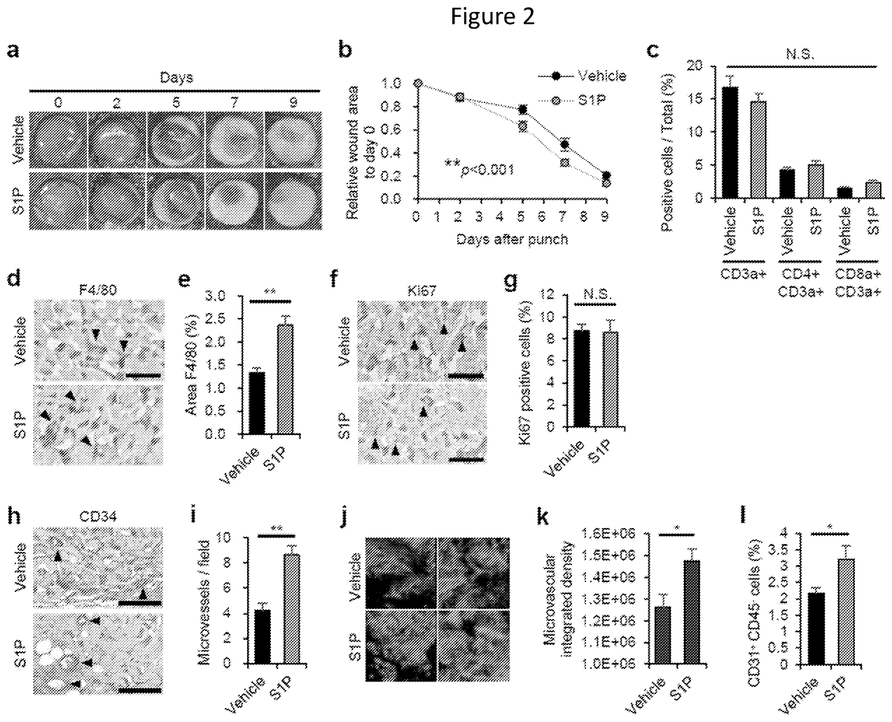

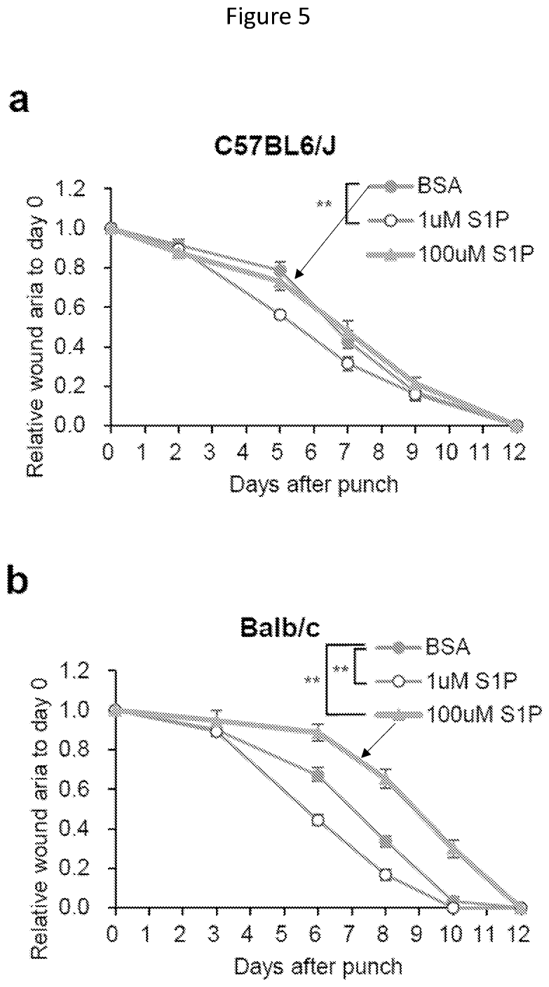

Next, we examined the effects by topical S1P treatment in mouse excisional wound splinting model. In wounds with treated with 1 μM S1P treatment, wound closure was significantly promoted compared to those treated with control vehicle treatment (FIG. 2a,b). On the other hand, wound closure with high concentration S1P (100 μM) treatment showed no difference comparing with those with vehicle treatment in C57BL6/J mice (FIG. 5a). Furthermore, wound closure with 100 μM S1P treatment was significantly obstructed in Balb/c mice (FIG. 5b). These results suggest that topical application with too high a concentration of S1P result in toxicity in wound. However, the mechanism of effective topical S1P treatment did not involve the effects we expected. For example, the percentage of T cells in wounds did not increase (FIG. 2c), and not change in macrophages was observed (FIG. 2d,e). Further, no effect in cell proliferation was induced (FIG. 2f,g). Our results in immunohistochemistry for CD34 showed that the mechanism of the treatment effects with S1P involved angiogenesis (FIG. 2h,i). We performed neovasculature analysis in Balb/c mice and confirmed the development of neovasculatures in scars with S1P treatment (FIG. 2j). Thus, this simple approach with topical S1P application for wound treatment acts effectively by promoting angiogenesis.

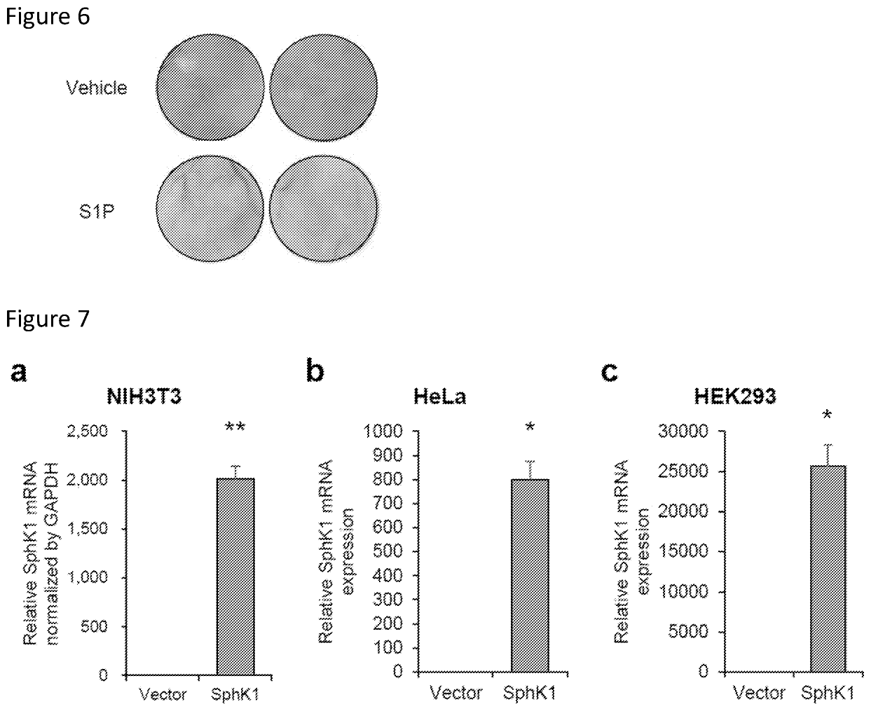

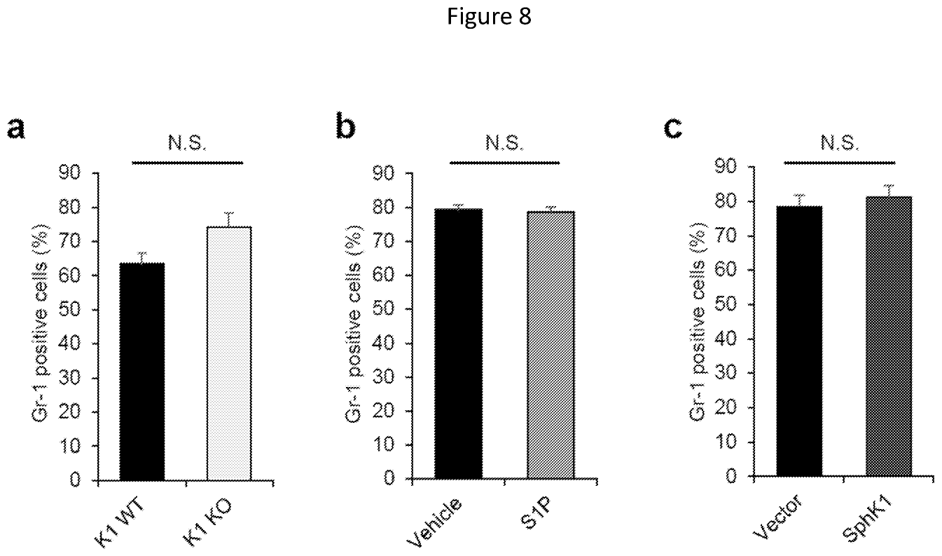

We also tested overexpression of SphK1 by topical approach to increase the S1P concentration stably in the local wound area. Wounds lacking an epidermis barrier are preferred for treating with topical gene delivery effectively, and suitable expression vectors encoding of SphK1 can be combined with nanoparticles as described above. We used super carbonate apatite (sCA) which is a safe biomaterial, and can be generated by simple methods with low cost ^{27,28}. We produced sCA capsuling vector or SphK1-expressing plasmids (Vector- or SphK1-sCA), and confirmed in vitro transfection efficiency (FIG. 6). Then, we applied an ointment including Vector- or SphK1-sCA in mouse wound splinting model (FIG. 3a). We could confirm the protein expressions of V5-tag in the wound surface tissues at two days after application, which showed that our in vivo topical transfection was successful (FIG. 3b). Wound closures treated with SphK1-sCA were promoted clearly in comparison with those treated with Vector-sCA (p<0.0001) (FIG. 3c,d). Also SphK1-sCA treatment did not influence granulocyte recruitment on day 2 after injury in inflammatory phase (FIG. 6). However, all of the percentages of CD3a⁺, CD4⁺CD3a⁺, or CD8a⁺CD3a⁺T cells in wounds significantly increased at day 5 (FIG. 3e). In addition, we observed significant increase in macrophages recruitment (FIG. 3f,g). Further, cell proliferation (FIG. 3h,i) and angiogenesis (FIG. 3j,k) in wounds treated with SphK1-sCA significantly increased. These results suggest the possibility that various kinds of wound-related factors increase widely due to application of SphK1-encoding plasmids. We collected wound surface tissues at day 5 to check the protein expressions of such factors. Western blots showed increased expressions of VEGF, FGF-2 or IGF-1, which are typical wound-related factors, in wounds treated with SphK1-sCA (FIG. 3I).

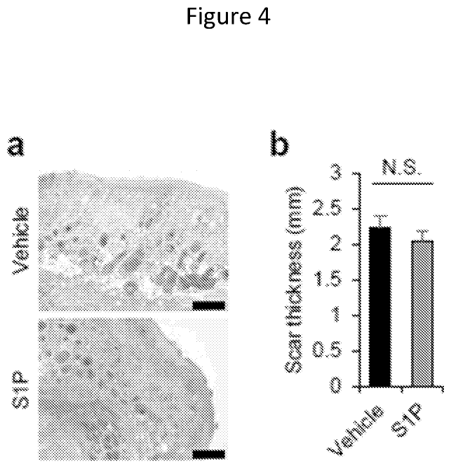

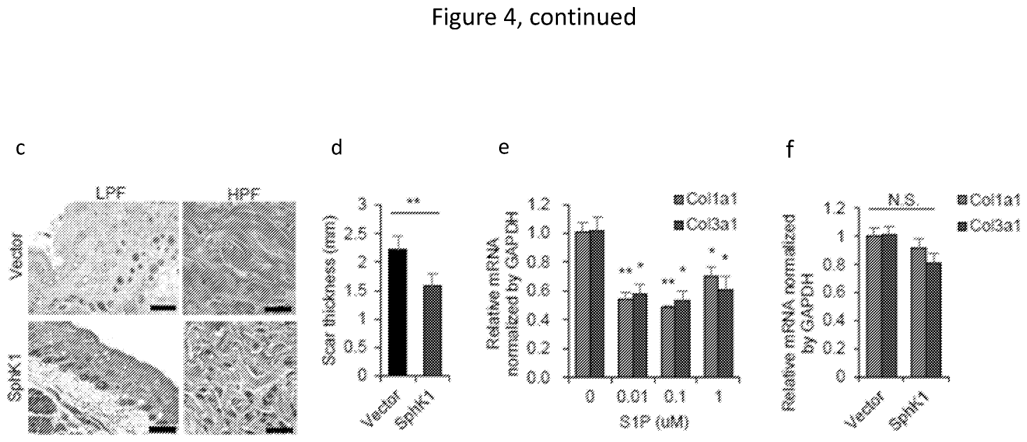

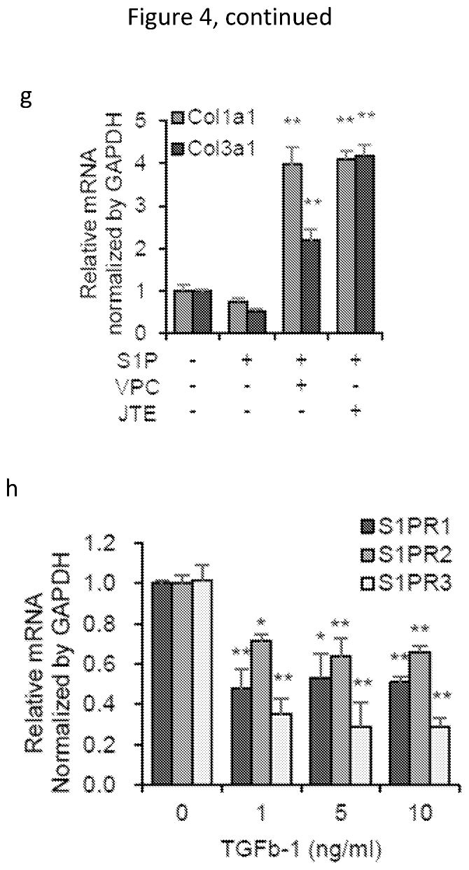

However, we unexpectedly noticed other effects of SphK1-sCA treatment. In particular, scar formation at the point when epithelization completed were clearly inhibited in wounds treated with SphK1-sCA. We analyzed scar thickness histologically. A statistically significant reduction in scar thickness were not obtained using S1P treatment (FIG. 4a,b). However, scar thicknesses was significantly thinner after SphK1-sCA treatment, and collagen bundles were also clearly thin in high magnificent images.

We investigated the roles of SphK1 and S1P signaling in collagen production in dermal fibroblasts to clarify the mechanism of this scarless wound healing. Transcription of Col1a1 and Col3a1 was inhibited in NIH 3T3 stimulated with exogenous S1P (FIG. 4c).

On the other hand, no difference were seen in collagen transcription between cells transfected with vector-sCA and those with SphK1-sCA (FIG. 4d). In addition, in cells stimulated with S1P in presence of S1PR1/3 inhibitor (VPC23019) or S1PR2 inhibitor (JTE013), collagen transcription increased under both inhibitor existence (FIG. 4e). In other words, not endogenous but exogenous S1P shows anti-fibrotic effect receptor-non-selectively in dermal fibroblasts. We ascertained that transcription of S1PRs was regulates in NIH 3T3 cells activated with TGFβ-1 (FIG. 4f). The anti-fibrotic effect of S1PR signaling is suppressed in proliferative phase of wound healing, and becomes effective as epithelization advance. This interaction provides the balance between tissue construction and inhibition of excessive fibrosis in the complex process of wound healing. Thus, and without intending to be bound by any particular theory, it appears that for rapid and scarless wound healing, exogenous S1P applied to wound surfaces is not enough and SphK1 gene delivery is preferred.

It will be recognized from the foregoing that the present disclosure provides innovative elements as new approach in wound treatment. In particular, topical SphK1 gene delivery is a successful approach to increase extended various wound-related factors in a proper balance. Second, the results demonstrate safe topical gene delivery with nanoparticles. Third, the disclosure demonstrates that rapid and scarless wound healing can be accomplished with only topical application.

The following materials and methods were used to obtain the foregoing results.

Mice

C57BL/6J and BALB/cJ mice were purchased from Jackson Laboratory. SphK1 KO mice and S1PR2 KO were from R. Proia. Animal procedures were approved by the Institutional Animal Care and Use Committee at Virginia Commonwealth University and the Animal Experimental Ethical Review Committee of Nippon Medical School.

Mouse Excisional Wound Splinting Model

Mouse excisional wound splinting model were generated as previously published¹. Mice were anesthetized using isoflurane and removed dorsal hair. Two of 5 mm-diameter full-thickness skin punches were created symmetrically besides the midline. 12 mm diameter circle-shaped silicon lubber splints in which 6 mm diameter circles were punched in center were used for wound splinting. Splints were fixed with instant-bonding adhesive and sutures around wounds. After application with any ointment, dressings were performed with Tegaderm (3M, Maplewood, Minn.).

Preparation of S1P Ointment

S1P was purchased from Sigma-Aldrich (Carlsbad, Calif.). 1 mM S1P in 4% bovine serum albumin was prepared with sonication, diluted 1000-fold for 1 μM ointment or 10-fold for 100 μM ointment with Aquaphor® and mixed well.

Plasmid Construction

Murine SphK1 gene was amplified using TaKaRa Ex Taq Hot Start Version (TaKaRa, Japan). We performed plasmid construction using pcDNA3.1/V5-His TOPO TA Expression Kit (Invitrogen, Carlsbad, Calif.).

Cell Culture

Mouse dermal fibroblast NIH 3T3 cells were cultured in DMEM. To analyze the production of collagens, ascorbic acid 2-phosphate (Sigma-Aldrich) was added in culture medium to 0.2 mM of final concentration.

In Vitro Transfection Using sCA

We performed sCA preparation as previously published (23). We mixed 4 μl of 1M CaCl2 with 2 μg of plasmid DNA in 1 mL of an inorganic solution (NaHCO3; 44 mM, NaH2PO4; 0.9 mM, CaCl2; 1.8 mM, pH 7.5), then incubated at 37° C. for 30 min. The solution was centrifuged at 12,000 rpm for 3 min, and the pellet was dissolved with DMEM. We sonicated the solution in a water bath for 10 min to generate sCA. Cells were cultured in 6 well plate for 24 hours, then incubated with sCA-DMEM solution for 6 hours. We changed medium into DMEM with 10% FBS and incubated for additional 48 hours, then collected for total RNA isolation.

Preparation of Plasmid-sCA Ointment

We generated sCA with 100 ug plasmid DNA and dissolved the sCA pellet with 50 μl PBS. Then we mixed all of the solution into 200 μl of Aquaphor®. We used 250 μl ointment for 4 wounds of 2 mice.

Wound Area Analysis

Digital photo images were analyzed using GIMP 2.8 software. The pixels of wound area were normalized by those of inside area of silicon splint. Then, the ratios devised by wound area at day 0 were calculated.

Flow Cytometry

We performed cell separation from mouse wound tissues as previously published⁴. We digested the tissues cut into small pieces in DMEM with 10% FBS, 1.2 mg/ml hyaluronidase (Sigma Aldrich), 2 mg/ml collagenase (Sigma Aldrich), and 0.2 mg/ml DNase I (Sigma Aldrich) at 37° C. for 90 min. Cell pellets were resuspended in PBS with 2% FBS, incubated with anti-CD16/32 antibody (Biolegend, San Diego, Calif.) for 5 min to block Fcγ receptors. For inflammatory cell recruitment analysis, we stained with phycoerythrin (PE)-conjugated anti-Gr-1, allophycocyanin (APC)-conjugated anti-CD3a, PE/CY7 conjugated anti-CD4, or fluorescein isothiocyanate (FITC)-conjugated anti-CD8a antibody (CiteAb, Bath, UK) at 4° C. for 20 min. For angiogenesis analysis, we stained with PE-conjugated anti-CD31 and FITC-conjugated anti-CD45 (Biolegend). Cells were analyzed with FACSDiva (BD, San Jose, Calif.).

Quantitative RT-PCR

Total RNA was extracted using TRIzol® Regent (Invitrogen). cDNA was then synthesized using High Capacity cDNA Reverse Transcription Kits (Applied Biosystems, Foster City, Calif.). qRT-PCR was performed using an CFX96 Real-Time System (Bio-Rad, Hercules, Calif.) with PowerUp SYBR Green master mix (Bio-Rad). GAPDH served as the internal control. Relative expression was calculated using the 2-ΔΔCt method with correction for different amplification efficiencies.

Immunohistochemistry

We performed H&E, Masson's trichrome staining on paraffin-embedded sections. We used primary antibodies against F4/80, Ki67 and CD34. Immunostaining was developed with VECTASTAIN Universal Elite ABC Kit (Vector, Burlingame, Calif.). We purchased all antibodies from Abcam (Cambridge, UK). We analyzed the results using ImageJ.

Neovasculature Analysis

We performed neovasculature analysis using standard approaches.

Photo acoustic imaging analysis for angiogenesis estimation

We performed photoacoustic imaging system using WEL5100 (Hadatomo™) (Advantest, Japan) for angiogenesis estimation as previously reported^{5,6}. We analyzed the images using ImageJ.

Western Blot

Wound tissue was homogenated with nitrogen liquid and total protein was isolated with 1% NP-40. Equal amounts of protein were separated on a SDS-PAGE and transferred to a nitrocellulose membrane. We purchased primary antibody against V5 from Invitrogen, VEGF, FGF-2 from Santa Cruz (Santa Cruz, Calif.), IGF-1 from Abcam, and GAPDH from Cell signaling (Danvers, Mass.), and horseradish peroxidase-conjugated IgG against mouse, rabbit, or goat from Jackson Immuno Research (West Grove, Pa.). The membranes were developed using SuperSignal Chemiluminescent Substrates (Thermo Fisher scientific, Cambridge, Mass.).

Scar Thickness Analysis

We calculated scar thickness with Masson's trichrome stain images using ImageJ.

Statistical Analysis

Comparisons between subjects were evaluated using the two-factor repeated measures ANOVA. Multiple comparisons were evaluated with post-hoc Tukey test. Comparisons between two groups were evaluated using Student's t-test or Welch's t-test after F test. P<0.05 was considered significant. All statistical analyses were performed using the Statce12 software (OMS, Japan).

Although the embodiments have been described in detail for the purposes of illustration, it is understood that such detail is solely for that purpose, and variations can be made therein by those skilled in the art without departing from the spirit and scope of the disclosure, embodiments of which are defined by the following claims.

### REFERENCES MENTIONED

1. Bryant, W. & J, F. Wound healing. Adv Dermatol 10, 77-96 (1995).
2. Boyce, D. E., Jones, W. D., Ruge, F., Harding, K. G. & Moore, K. The role of lymphocytes in human dermal wound healing. Br. J. Dermatol. 143, 59-65 (2000).
3. Ploeger, D. T. et al. Cell plasticity in wound healing: paracrine factors of M1/M2 polarized macrophages influence the phenotypical state of dermal fibroblasts. Cell Commun. Signal. 11, 29 (2013).
4. Spiegel, S. & Milstien, S. Sphingosine-1-phosphate: an enigmatic signalling lipid. Nat. Rev. Mol. Cell Biol. 4, 397-407 (2003).
5. Hait, N. C., Oskeritzian, C. A., Paugh, S. W., Milstien, S. & Spiegel, S. Sphingosine kinases, sphingosine 1-phosphate, apoptosis and diseases. Biochem. Biophys. Acta 1758, 2016-2026 (2006).
6. Aoki, M., Aoki, H., Ramanathan, R., Hait, N. C. & Takabe, K. Sphingosine-1-Phosphate Signaling in Immune Cells and Inflammation: Roles and Therapeutic Potential. Mediators Inflamm. 2016, (2016).
7. Moreno, E. et al. Assessment of b-lapachone loaded in lecithin-chitosan nanoparticles for the topical treatment of cutaneous leishmaniasis in L. major infected BALB/c mice. Nanomedicine 11, 2003-2012 (2015).
8. Stein, C. & Küchler, S. Targeting inflammation and wound healing by opioids. Trends Pharmacol. Sci. 34, 303-312 (2013).
9. Hofmann, U. et al. Activation of CD4+T lymphocytes improves wound healing and survival after experimental myocardial infarction in mice. Circulation 125, 1652-1663 (2012).
10. Liu, M. et al. 12-Hydroxyheptadecatrienoic acid promotes epidermal wound healing by accelerating keratinocyte migration via the BLT2 receptor. J. Exp. Med. 211, 1063-78 (2014).
11. Watterson, K. R., Lanning, D. A., Diegelmann, R. F. & Spiegel, S. Regulation of fibroblast functions by lysophospholipid mediators: potential roles in wound healing. Wound Repair Regen. 15, 607-616 (2011).
12. Takabe, K. & Spiegel, S. Export of sphingosine-1-phosphate and cancer progression. J. Lipid Res. 55, 1839-1846 (2014).
13. Nagahashi, M. et al. Sphingosine-1-phosphate produced by sphingosine kinase 1 promotes breast cancer progression by stimulating angiogenesis and lymphangiogenesis. i Cancer Res. 72, 726-735 (2012).
14. Liang, J. et al. Sphingosine-1-phosphate links persistent STAT3 activation, chronic intestinal inflammation, and development of colitis-associated cancer. Cancer Cell 23, 107-20 (2013).
15. Nagahashi, M. et al. Sphingosine-1-phosphate in chronic intestinal inflammation and cancer. Adv. Biol. Regul. 29, 997-1003 (2012).
16. Maceyka, M. et al. SphK1 and SphK2, sphingosine kinase isoenzymes with opposing functions in sphingolipid metabolism. J. Biol. Chem. 280, 37118-37129 (2005).
17. Takabe, K., Paugh, S. W., Milstien, S. & Spiegel, S. ‘Inside-out’ signaling of sphingosine-1-phosphate: therapeutic targets. Pharmacol. Rev. 60, 181-195 (2008).
18. Matloubian, M. et al. Lymphocyte egress from thymus and peripheral lymphoid organs is dependent on S1P receptor 1. Nature 427, 355-360 (2004).
19. Schwab, S. R. & Cyster, J. G. Finding a way out: lymphocyte egress from lymphoid organs. Nat. Immunol. 8, 1295-1301 (2007).
20. Schwab, S. R. et al. Lymphocyte sequestration through S1P lyase inhibition and disruption of S1P gradients. Science (80-.). 309, 1735-1739 (2005).
21. Pappu, R. et al. Promotion of lymphocyte egress into blood and lymph by distinct sources of sphingosine-1-phosphate. Science (80-.). 316, 295-298 (2007).
22. Lee, M. J. et al. Vascular endothelial cell adherens junction assembly and morphogenesis induced by sphingosine-1-phosphate. Cell 99, 301-312 (1999).
23. Allende, M. L. & Proia, R. L. Sphingosine-1-phosphate receptors and the development of the vascular system. Biochim. Biophys. Acta 1582, 222-227 (2002).
24. Ledgerwood, L. G. et al. The sphingosine 1-phosphate receptor 1 causes tissue retention by inhibiting the entry of peripheral tissue T lymphocytes into afferent lymphatics. Nat. Immunol. 9, 42-53 (2008).
25. Allende, M. L. et al. Mice deficient in sphingosine kinase 1 are rendered lymphopenic by FTY720. J. Biol. Chem. 279, 52487-52492 (2004).
26. Bandhuvula, P. & Saba, J. D. Sphingosine-1-phosphate lyase in immunity and cancer: silencing the siren. Trends Mol. Med. 13, 210-217 (2007).
27. Chowdhury, E. & Akaike, T. High performance DNA nano-carriers of carbonate apatite: Multiple factors in regulation of particle synthesis and transfection efficiency. Int. J. Nanomedicine 2, 101-106 (2007).
28. Wu, X. et al. Innovative Delivery of siRNA to Solid Tumors by Super Carbonate Apatite. PLoS One 10, (2015).

1. A method for reducing scarring during healing of a tissue wound comprising topically applying to the wound a composition comprising sphingosine-1-phosphate (S1P), and/or an expression vector that encodes sphingosine kinasel (SphK1).

2. The method of claim 1, wherein the composition comprises the expression vector, the composition further comprising biocompatible nanoparticles.

3. The method of claim 2, wherein the biocompatible nanoparticles comprise super carbonate apatite (sCA).

4. The method of claim 1, wherein scarring in the wound is reduced relative to a control, wherein the control comprises a value from wound healing in the absence of exogenously applied S1P and/or an absence of the expression vector.

5. The method of claim 4, wherein scarring in the wound is reduced relative to a control, wherein the control comprises a value from wound healing in the absence of exogenously applied S1P and/or an absence of the expression vector.

6. The method of claim 4, wherein the reducing of the scarring comprises inhibition of keloid formation.

7. The method of claim 4, wherein the inhibition comprises prevention of keloid formation.

8. The method of claim 1, wherein the composition comprises an ointment.

9. A composition for use in performing a method of claim 1, the composition comprising sphingosine-1-phosphate (S1P), and/or an expression vector that encodes sphingosine kinasel (SphK1).

10. The composition of claim 9 comprised by an ointment.

11. The composition of claim 9 comprising the expression vector.

12. The composition of claim 9 comprising the S1P.

13. An article of manufacture comprising a composition of claim 9, the article comprising packaging, the packaging comprising printed material providing instructions for using the composition and an indication that the composition is for use in healing of wounds.

14. The article of manufacture of claim 13, wherein the composition is comprised by an ointment.

15. The article of manufacture of claim 13 comprising the expression vector.

16. The article of manufacture of claim 13 comprising the S1P.

17. The article of manufacture of claim 13, further comprising a device used for wound healing.

18. A device comprising a composition of claim 9.

19. The device of claim 18, wherein the device is selected from a wound dressing, a suture, and a staple.

20. The device of claim 19, wherein the device is a wound dressing.
